(19) United States
(12) Patent Application Publication
(10) Pub. No.: US 2005/0112603 A1
(43) Pub. Date: May 26, 2005

(54) Method for purification, modification and immobilization of recombinant protein

(75) Inventors: Tai Chih Kuo, Taipei (TW); Han Jung Lin, Taoyuan County (TW)

Correspondence Address: BACON & THOMAS, PLLC, 625 SLATERS LANE, FOURTH FLOOR, ALEXANDRIA, VA, 22314, US

(73) Assignee: Industrial Technology Research Institute, Hsin Chu (TW)

(21) Appl. No.: 10/798,440

(22) Filed: Mar. 12, 2004

(30) Foreign Application Priority Data
Nov. 21, 2003 (TW) 92132715

(51) Int. Cl. B05D 3/00; C12Q 1/68; C12P 21/06; C12N 9/10; C12M 1/34

(52) U.S. Cl. 435/6; 435/193; 435/287.2; 427/2.11

(57) ABSTRACT

The present invention relates to a method for purifying, modifying and immobilizing recombinant protein. The method utilizes genetic engineering to tag a DNA sequence encoding a target protein with a specific tag and express the vector to obtain a recombinant protein. The recombinant protein is then purified and modified by an affinity column and modification reagent. After exchanging the recombinant protein with a decoupling reagent, the recombinant protein is immobilized onto a specific substrate. The method, combining steps of purification, modification and immobilization, provides convenience to using recombinant protein. The omission of the use of dialysis or molecular sieve leads to a shorter period dedicating for removing excessive reagents and the increase of efficiency of recombinant protein recovery.

### BACKGROUND OF THE INVENTION

1. Field of the Invention

The present invention relates to a method for purifying, modifying and immobilizing recombinant protein. The method combines steps of purification, modification and immobilization so as to omit the step of dialysis or molecular sieve purification. This leads to a shorter period dedicating for removing excessive reagents, and also increasing the efficiency of recombinant protein recovery as well as facilitating the immobilization of the recombinant protein on a subtrate.

2. Description of the Related Arts

As the human genetic map being established, more and more proteins will be decoded. In order to efficiently determine and analyse protein function, techniques in protein purification, modification and immobolization have been vigorously developed.

Traditional methods for purification employ various chromatography. Chromatography differentiates between different proteins by their different properties (eg., isoelectric point, molecular weight). However, chromatography based on isoelectric point or molecular weight has low specificity, resulting in poor differentiation, and is often accompanied with sample dilution and loss during the separation step. To solve this problem, methods have been developed to tag the target protein with a specific tag by genetic engineering to allow the protein attach to a column specific to the tag and therefore be isolated from other impurities. The purified protein is then exchanged from the affinity column with a decoupling reagent. The method has been increasingly applied for purifying recombinant protein. Conventional specific tags for proteins, such as Histidine tag, is used with an affinity column that specifically captures the tag ( eg., a Ni-IDA column for Histidine tag) to isolate the protein from other impurities. The protein is then exchanged from the column using a decoupleing reagent according to the specific tag (eg., immidazole for Histidine tag). This method is more specific comparing with the traditional one, it effectively solves the problem of poor isolation of the traditional chromatography technique.

To increase the efficiency of utilization and recycling of the purified protein, protein is immobilized on a subject so as to prevent protein loss during the analysation. Traditionally, this is achieved by modifiying the subtrate so its sufrace has a specific functional group. This functional group binds to the branch functional groups of amino acid residues of the protien, and therefore immobilizes the protein. However, this method often leads to the loss of protein function, and the binding is usually irreversible.

The method based on the specific and strong binding between biomolecules instead of chemical bonding for immobilization has been increasingly used. Taking biotin and streptavidin as an example, since biotin can be added to various biomolecules (eg., DNA and protein), with a substrate coated with streptavidin, biomolecules can be immobilized on the substrate for various applications.

However, both the steps of exchanging protein from the column by a decoupling reagent and the biotinylation of the protein need to remove the decoupling reagent and unreacted reagents by dialysis or molecular sieve purification. These are not ideal procedures. Dialysis has three major shortcomes: (1)inconvenient: large amount of dialytic solution needs to be prepared, and the work becomes tedious when multiple samples are to be dialyzed at the same time; (2)time-consuming: it requires at least 2-3 times of balance, each takes at least 4-6 hours, and therefore it takes at least one day to complete a dialysis; (3)low recovery of protein: protein may denature or attach to the dialysis membrane. Molecular sieve purification has two main problems: (1)small volumn of the column: only one-tenth column volumn of sample can be processed in the molecular sieve column each time; (2)low recovery and dilution: due to changes of salt concentration within the column during the elution process, protein will attache to the column and diluted by the elution solution. Therefore, it is an important issue to improve protein recovery rate during the purification and modification steps.

U.S. Pat. No. 6,150,123 desclosed a method of in-column biotinylation. An affinity columnn is used to capture a recombinant protein, and the protein is then biotinylated within the column. In fact, as long as the recombinant protein capturing reaction does not clash with the biotinylation reaction, the biotinylation reaction is not restricted to be carried out within the column. The biotinylation can be carried out in a liquid solution before the capturing of the recombinant protein by the affinity column.

As for the capturing of recombinant protein using a affinity column, U.S. Pat. No. 6,150,123 discloses a method based on the specificity of ligand-recepter binding, such as target protein and its monoclonal antibody, glycoprotein and the lectin, and enzyme and the substrate. The method immobilizes the ligand in the column, and captures the corresponding receptor by the ligand-receptor affinity. Although this method is specific, the antibody or natural ligand of the target protein need to be obtained before purifying the target protein. When two or more proteins are purified at the same time, the purification of the antibodies or natural ligands will complicate the practice, increase the required time and resources. Therefore this is not a convenient and widely-applicable method.

### SUMMARY OF THE INVENTION

In consideration of the problems of the prior art, the present invention provides a method for purifying, modifying and immobilizing recombinant protein. The method utilizes genetic engineering to tag the target protein and express the recombinant protein. The recombinant protein is then purified and modified by affinity column and modification reagent. After exchanging the recombinant protein with a decoupling reagent, the recombinant protein is immobilized onto a specific substrate.

The present invention provides a method for purifying, modifying and immobilizing recombinant protein. The method comprises the steps of: tagging the DNA sequence encoding a target protein into a recombinant vector with a specific tag sequence; expressing the vector under suitable condition to obtain a recombinant protein; purifying and modifying said recombinant protein by using an affinity column and a modification reagent; exchanging said recombinant protein which has been attached to the affinity column with a decoupling reagent; and immobilizing said recombinant protein onto a substrate.

The specific tag comprises Histidine tag, Maltose-binding tag, or Glutathion-S-Transferase fusion protein (GST tag).

The gene expression system for expressing the recombinant protein utilizes prokaryotic cell, eukaryotic cell or in vitro transcription/translation system.

According to the above gene expression system of the present invention, the prokaryotic cell is E coli. and the eukaryotic cell is yeast, insect cell or mammalian cell.

The affinity column for capturing the recombinant protein is chosen in corresponding to said specific tag.

When the specific tag is Histidine tag, a metal chelation column is used as the affinity column. When the specific tag is Maltose-binding tag, an amylose column is used as the affinity column. When the specific tag is GST tag, a glutathione column is used as the affinity column.

The metal chelation column is represented by a general formula as metal-X column. The “metal” in said formula used herein refers to the metal having affinity attraction with histidine tag, said metal comprises, but not limited to, nickel(Ni), zinc(Zn), cobalt(Co), or copper(Cu). The “X” in said formula used herein refers to the matter that can chelate with the aforementioned metal, said X comprises, but not limited to, iminodiacetic acid(IDA) nitrilotriacetic acid(NTA), tris(carboxymethyl)ethylendiamin, carboxymethylaspartate, or TALON.

The aforementioned metal-X column, preferably is nickel iminodiacetic acid(Ni-IDA) or copper iminodiacetic acid(Cu-IDA).

The modification of the recombinant protein utilizes a biotinylation reaction to add a biotin functional group to the recombinant protein.

The biotinylation of the recombinant protein can be carried out before the purification of the recombinant protein, or it can be carried out in the affinify column after the recombinant protein being captured in the column.

The modified and purified recombinant protein is exchanged from the affinity column by a decoupling reagent. The decoupling reagent is a compound or protein that is capable of eluting the recombinant protein from the affinity column. The decoupling reagent is chosen according to the sequence of the specific tag and the properties of the affinity column. For example, when the specific tag is Histidine tag, the decoupling reagent can be immidazole. When the specific tag is maltose-binding tag, the decoupling reagent can be maltose. When the specific tag is GST tag, the decoupling reagent can be glutathione.

The immobilization of the recombinant protein is based on the binding between the coating of substrate and the modifying functional group added to the recombinant protein. When the recombinant protein is carries a biotin functional group, the coating of substrate is streptavidin. The substrate comprises metal (eg., iron beads),glass or polymers.

The recombinant protein immobilized on the substrate can be filtrated or washed to remove the decoupling reagent in the solution.

The present invention provides a method for purifying, modifying and immobilizing recombinant protein. The method comprises utilizing genetic engineering to tag a DNA sequence encoding a target protein with a specific tag so that the target protein can be retained within a affinity column which specifically recognizes the tag. The modification of the protein (eg., biotinylation) can be carried out in a solution before the protein goes through the affinity column, or it can be carried out in the affinity column after the protein being captured. The recombinant protein is then exchanged from the column by a decoupling reagent. Finally, the decoupling reagent can be removed during the immobolization process. The method of the present invention solves the problems of the time-consuming and low recovery yield when dialysis or molecular sieve is used. Morever, the method is not limited to single recombinant protein applications, it is capable of processing multiple recombinant proteins at the same time. Since different recombinant proteins carry the same specific tag, only a single step of a single affinity column is required for the recognition and capturing of various recombinant proteins. The method of the present invention efficiently improves the limitation of single ligand corresponding to single receptor in the prior art (eg., U.S. Pat. No. 6,150,123) and is capable of purifying multiple proteins at the same time.

### BRIEF DESCRIPTION OF THE DRAWINGS

The present invention will be more fully understood and further advantages will become apparent when reference is made to the following description of the invention and the accompanying drawings in which:

FIG. 1 is a flow chart showing the method for purifying, modifying and immobilizing recombinant protein of the present invention.

FIG. 2 is a flow chart showing the method for purifying, modifying and immobilizing recombinant protein of Example 1 of the present invention.

FIG. 3 is a diagram showing the results of the electrophroesis of the purifying, modifying and immobilizing steps of Example 1 of the present invention.

FIG. 4 is a flow chart showing another method for purifying, modifying and immobilizing recombinant protein of Example 2 of the present invention.

FIG. 5 is a diagram showing the results of the electrophroesis of the purifying, modifying and immobilizing steps of Example 2 of the present invention.

### DETAILED DESCRIPTION OF THE INVENTION

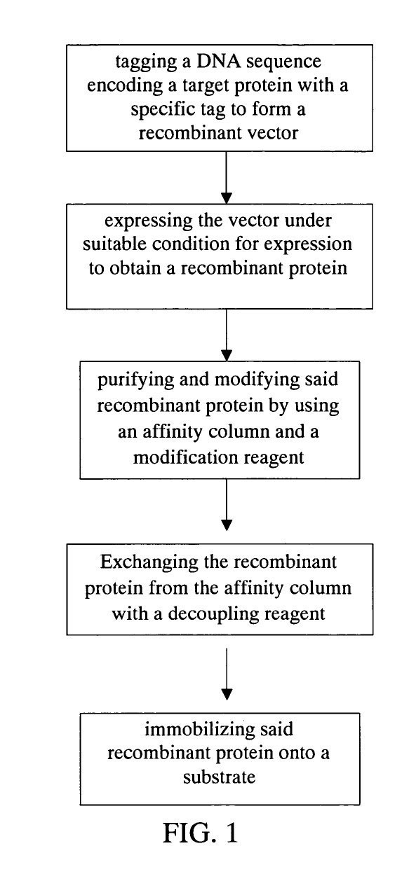

In order to rapidly modify and immobolize the recombinant protein on the substrate, the present invention provides a method for purifying, modifying and immobilizing recombinant protein. As shown in FIG. 1, the method comprises the following steps: tagging a DNA sequence encoding a target protein with a specific tag to form a recombinant vector and expressing the vector under suitable condition to obtain the recombinant protein; purifying and modifying recombinant protein by affinity column and modification reagent; exchanging the attached recombinat protein from the affinity column by a decoupling reagent; and immobilizing the recombinant protein onto a substrate. The detail of each step will be described as follows. Firstly, in an inducible gene expression vector, a specific tag sequence is added to the 5′ or 3′ end of the target protein. Conventional specific tag sequences include Histidine tag, maltose-binding tag, and GST tag. The vector is then introduced into an expression system to produce the recombinant protein in large quantity. There are two ways to modify and purify recombinant protein with a specific tag. First, the recombinant protein is modified in a solution, and then is passed through an affinity column which will retain the recombinant protein specifically via the tag. Alternatively, the recombinant protein is firstly passed through the column,and the captured recombinant protein is then modified within the column.

The affinity column for capturing the recombinant protein is chosen according to the specific tag. When the specific tag is Histidine tag, a metal chelating column is used as the affinity column. When the specific tag is Maltose-binding tag, an amylose column is used as the affinity column. When the specific tag is GST tag, a glutathione column is used as the affinity column.

The modification of the recombinant protein means adding a molecule to the recombinant protein. The molecule has specific affinity with another molecule. An example is biotin and streptavidin. Biotin can be added to many kinds of biomolecules, including DNA and protein. The specific and strong binding between biotin and streptavidin has been increasingly applied to molecular biology researches.

After the recombinant protein is captured in the affinity column and therefore is isolated from other impurities, excess modifying reagent is washed away by a buffer. The modified and purified recombinant protein is then exchanged from the affinity column by a decoupling reagent. The decoupling reagent is chosen according to the combined properties of the specific tag and the affinity column. When the specific tag is Histidine tag and the affinity column is a metal chelating column, the decoupling reagent is immidazole. When the specific tag is a maltose-binding tag and the affinity column is an amylose column, the decoupling reagent is maltose. When the specific tag is GST tag and the affinity column is glutathione column, the decoupling reagent can be glutathione.

After exchanging the recombinant protein from the affinity column with the decoupling reagent, the decoupling reagent in the protein solution can be removed by the wash step after the immobilization of the recombinant protein. The immobolization of the recombinant protein is carried out by coating a substrate with a substance which will forms affinity bond with the modifying molecule of the protein. For example, when the modifying molecule of the recombinant protein is biotin, the surface of the substrate is coated with streptavidin.

Without intending to limit it in any manner, the present invention will be further illustrated by the following preferred embodiments.

### EXAMPLE 1

### The Purification, Biotinylation, and Immobolization of Recombinant Thrombospondin N-Terminal Like Domain of Human Collagen Type 21 Expressed in E. coli

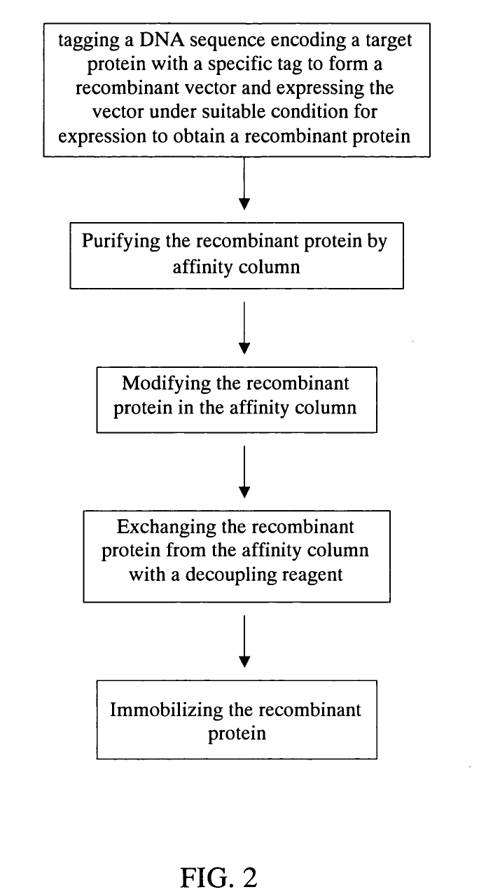

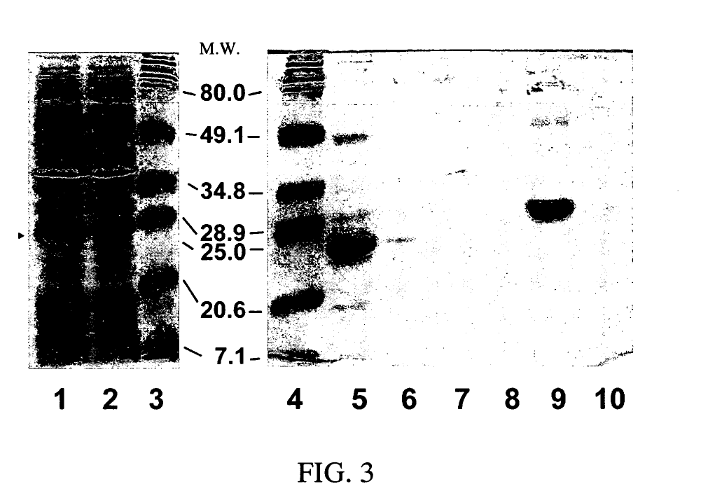

Example 1 is a method for purifying, modifying and immobilizing recombinant protein of the present invention according to the flow chart shown in FIG. 2. E. coli BL21De3 was used as the expression host for the pET28A (Novagen) vector containing the recombinant gene. Incubating with LB media containing the inducer, IPTG (1 mM), E. coli expressed the designed recombinant thrombospondin N-terminal like domain (TSPN like domain) of human collagen type 21 containing a Histidine tag. As shown in FIG. 3, comparing with Lane 2 (without IPTG induction), Lane 1 (with IPTG induction) showed the recombinant TSPN like domain at where the arrow is pointing. Lane 3 and Lane 4 were the molecular weight markers. Lane 5 showed the primarily purified recombinant TSPN like domain. Its molecular weight is 25 kD, which conformed the expected total molecular weight of TSPN like domain and Histidine tag. The recombinant TSPN like domain solution was then passed through a Ni-IDA affinity chromotography column. The recombinant TSPN like domain having the Histidine tag was selectively retained in the column. The solution passing out the column contained almost no recombinant TSPN like domain (Lane 6). Biotinylation reagent, NHS-LC-biotin, was then added to modify the recombinant TSPN like domain in the column so to attach biotin to the protein. This reaction would not effect the specific attachment of the recombinant TSPN like domain to the affinity column, and therefore the solution passing out the column did not contain the recombinant TSPN like domain (Lane 7). After the reaction was completed, the column was washed with buffer to remove unreacted biotinylation reagent. The buffer passing out the column also did not contain the recombinant TSPN like domain (Lane 8). Next, a decoupling reagent (Immidazole) was used to exchange the recombinant TSPN like domain from the affinity column. Since the recombinant TSPN like domain now carried the biotin, the molecular weight had increased (Lane 9). Mixed the recombinant TSPN like domain modified with biotin and iron beads modified with streptavidin. When biotin binded tightly to streptavidin, the iron beads was removed from the solution by a magnet. The solution was now free of the recombinant TSPN like domain (Lane 10).

### EXAMPLE 2

### The Purification, Biotinylation, and Immobolization of Recombinant Thrombospondin N-Terminal Like Domain of Human Collagen Type 21 Expressed in E. coli

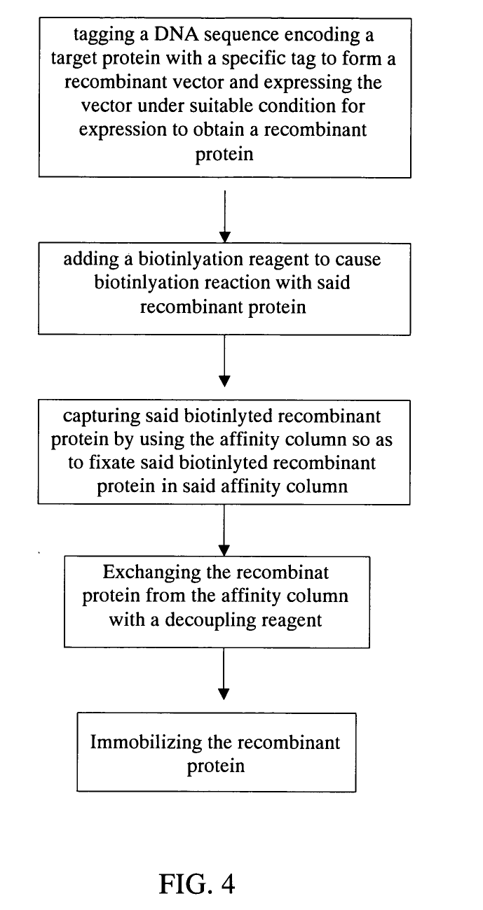

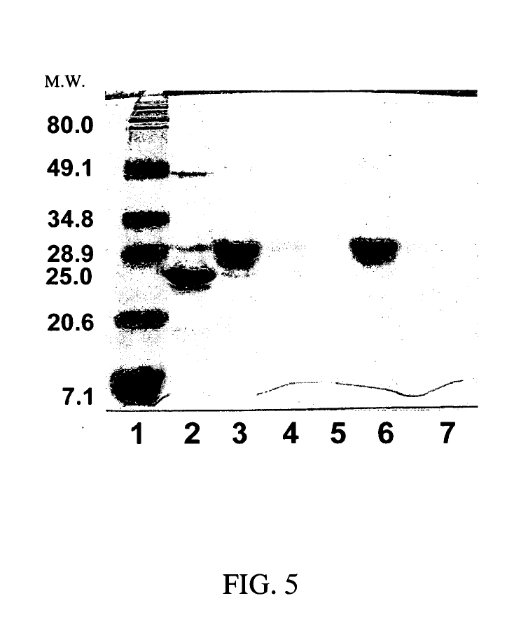

As shown in FIG. 4, the procedure was identical with that of Example 1 (FIG. 2) except that the biotinylation of the recombinant TSPN like domain was carried out earlier in a solution. The results of the present Example is shown in FIG. 5. Lane 1 was the molecular weight marker. The primarily purified recombinant TSPN like domain (Lane 2) was biotinylated in a solution so to have biotin molecules. The molecular weight of the recombinant protein therefore increased slightly (Lane 3). The biotinylated recombinant TSPN like domain was then passed through a Ni-IDA affinity chromatography column. The recombinant TSPN like domain was selectively retained in the column through its Histidine tag. The solution passing out the column contained very low amount of the recombinant TSPN like domain (Lane 4). The column was then washed with buffer to remove unreacted biotinylation reagent. The buffer passing out the column also did not contain the recombinant TSPN like domain (Lane 5). Next, a decoupling reagent (Immidazole) was used to exchange the recombinant TSPN like domain from the affinity column (Lane 6). Iron beads coated with streptavidin was added to the recombinant TSPN like domain solution. When biotin had binded tightly to streptavidin and the immobolization was completed, the iron beads was removed from the solution by a magnet. The solution was now free of the recombinant TSPN like domain (Lane 7).

While the invention has been particularly shown and described with the reference to the preferred embodiments thereof, the preferred embodiments does not limit the scope of the present invention in anyway. It will be understood by those skilled in the art that various changes in form and details may be made without departing from the spirit and scope of the invention. The scope of the present invention is defined as the following claims.

1. A method for fabricating a substrate by purification, modification and immobilization of recombinant protein, said method comprising the steps of:
tagging a DNA sequence encoding a target protein into a recombinant vector with a specific tag sequence;
expressing the vector under suitable condition to obtain a recombinant protein; purifying and modifying said recombinant protein by using an affinity column and a modification reagent;
exchanging said recombinant protein which has been attached to the affinity column with a decoupling reagent; and
immobilizing said recombinant protein onto a substrate.

2. The method as claimed in claim 1, wherein said specific tag comprises Histidine tag, Maltose-binding tag, or glutathione S transferase tag.

3. The method as claimed in claim 2, wherein said specific genetic tag is Histidine tag.

4. The method as claimed in claim 1, wherein said recombinant protein is prepared by using prokaryotic cell, eukaryotic cell or an in vitro transcription/translation system.

5. The method as claimed in claim 4, wherein said prokaryotic cell is E. coli.

6. The method as claimed in claim 4, wherein said eukaryotic cell is yeast, insect cell or mammalian cell.

7. The method as claimed in claim 1, wherein the affinity column for capturing the recombinant protein is chosen in corresponding to said specific tag.

8. The method as claimed in claim 7, when said specific tag is Histidine tag, a metal chelating column is used as the affinity column.

9. The method as claimed in claim 8, wherein the metal chelation column is represented by a general formula as metal-X column.

10. The method as claimed in claim 9, wherein the metal in said formula comprises nickel, zinc, copper, or cobalt.

11. The method as claimed in claim 9, wherein the X in said formula comprises iminodiacetic acid, nitrilotriacetic acid, tris(carboxymethyl)-ethylendiamin, carboxymethylaspartate, or TALON, a immobilized metal affinity resin.

12. The method as claimed in claim 9, wherein the metal-X column is Ni-iminodiacetic acid column or Cu-iminodiacetic acid column.

13. The method as claimed in claim 7, when said specific tag is Maltose-binding tag, an amylose column is used as the affinity column.

14. The method as claimed in claim 7, when said specific tag is a GST-tag, glutathione column is used as the affinity column.

15. The method as claimed in claim 1, wherein said recombinant protein is modified by using a biotinylation reaction so to add biotin functional groups to said recombinant protein.

16. The method as claimed in claim 15, wherein the modification of said recombinant protein comprising the steps of:
obtaining a solution containing the recombinant protein;
adding a biotinlyation reagent to cause biotinlyation reaction with said recombinant protein; and
capturing said biotinlyted recombinant protein by using the affinity column so as to fixate said biotinlyted recombinant protein in said affinity column.

17. The method as claimed in claim 15, wherein the modification of said recombinant protein comprising the steps of:
obtaining a solution containing the recombinant protein;
capturing said recombinant protein by using the affinity column so as to fixate said recombinant protein in said affinity column; and
adding a biotinlyation reagent to said affinity column to cause biotinlyation reaction with said recombinant protein fixated in said affinity column.

18. The method as claimed in claim 16, wherein said recombinant protein is exchanged from the affinity column by a decoupling reagent, said decoupling reagent is chosen according to the properties of the specific tag and the affinity column.

19. The method as claimed in claim 18, when said specific tag is Histidine tag and the affinity column is a metal chelating column, the decoupling reagent is imidazole.

20. The method as claimed in claim 18, when said specific tag is maltose-binding tag and the affinity column is an amylose column, the decoupling reagent is maltose.

21. The method as claimed in claim 18, when said specific tag is GST tag and the affinity column is a glutathione column, the decoupling reagent is glutathione.

22. The method as claimed in claim 1, wherein the immobilization of said recombinant protein is achieved by modifying the recombinant protein with biotin and attaching the biotin-modified recombinant protmemprotein on a substrate coated with streptavidin.
